(19) United States
(12) Patent Application Publication
(10) Pub. No.: US 2024/0249453 A1
(43) Pub. Date: Jul. 25, 2024

(54) SYSTEMS AND METHODS FOR ATTENUATION COMPENSATION IN SINGLE-PHOTON EMISSION COMPUTED TOMOGRAPHY (SPECT)

(71) Applicant: Washington University, St. Louis, MO (US)

(72) Inventors: Abhinav Kumar Jha, St. Louis, MO (US); Zitong Yu, St. Louis, MO (US); Md Ashequr Rahman, St. Louis, MO (US)

(21) Appl. No.: 18/416,578

(22) Filed: Jan. 18, 2024

Related U.S. Application Data
(60) Provisional application No. 63/480,576, filed on Jan. 19, 2023.

(51) Int. Cl. G06T 11/00; A61B 6/00; A61B 6/03

(52) U.S. Cl. CPC G06T 11/006; A61B 6/037; A61B 6/5282; G06T 2210/41; G06T 2211/441

(57) ABSTRACT

A system for single-photon emission computed tomography (SPECT) is provided. The system includes a computer device comprises at least one processor in communication with at least one memory device. The at least one processor is programmed to: a) store a model trained to generate an attenuation map of a subject being examined; b) receive a scatter-energy window projection of a first subject to be examined; c) execute the model with the scatter-energy window projection of the first subject as an input, wherein the model generates an attenuation map; d) receive a photopeak-energy window projection of the first subject to be examined; and e) perform attenuation compensation on the photopeak-energy window projection using the generated attenuation map.

### CROSS REFERENCE TO RELATED APPLICATIONS

This application claims priority to U.S. Provisional Patent Application 63/480,576, filed, Jan. 19, 2023, which is hereby incorporated by reference in its entirety.

### STATEMENT REGARDING FEDERALLY SPONSORED RESEARCH & DEVELOPMENT

This invention was made with government support under EB024647, EB031051, EB022827, EB031962 awarded by the National Institutes of Health. The government has certain rights in the invention.

### BACKGROUND

The present disclosure generally relates to computer-aided systems and methods of performing single-photon emission computed tomography (SPECT) imaging, and more specifically to performing attenuation compensation in SPECT imaging without the use of a computed tomography (CT) scan.

Myocardial perfusion SPECT (MPS) has an important and well-validated role in the diagnosis of coronary artery disease. Attenuation compensation is known to improve performance visual interpretation tasks in MPS. However, performing attenuation compensation requires the presence of an X-ray CT map, for which an additional CT scan of the patient is acquired, which is typically acquired with a SPECT/CT scanner. However, a major component of the SPECT marketshare (75-80% based on a market report from 2017) consists of SPECT only systems that do not have a CT component. This includes mobile SPECT systems that enable SPECT at remote locations.

The inclusion of CT also leads to multiple other disadvantages such as increased radiation dose, higher costs, patient inconvenience, and requirement of a CT scanner. Further, there is a possibility of misalignment between the SPECT and the CT scans, which can then lead to inaccurate diagnosis.

Due to all these reasons, there is an important need for methods to perform attenuation compensation without requiring the CT scan.

### BRIEF DESCRIPTION

In a first aspect, a system for single-photon emission computed tomography (SPECT) is provided. The system includes a computer device comprises at least one processor in communication with at least one memory device. The at least one processor is programmed to: a) store a model trained to generate an attenuation map of a subject being examined; b) receive a scatter-energy window projection of a first subject to be examined; c) execute the model with the scatter-energy window projection of the first subject as an input, wherein the model generates an attenuation map; d) receive a photopeak-energy window projection of the first subject to be examined; and e) perform attenuation compensation on the photopeak-energy window projection using the generated attenuation map. The system may include additional, less, or alternate functionality, including that discussed elsewhere herein.

In a second aspect, a method for single-photon emission computed tomography (SPECT) is provided. The method is implemented by a computer device comprising at least one processor in communication with one or more memory devices. The method includes: a) storing a model trained to generate an attenuation map of a subject being examined; b) receiving a scatter-energy window projection of a first subject to be examined; c) executing the model with the scatter-energy window projection of the first subject as an input, wherein the model generates an attenuation map; d) receiving a photopeak-energy window projection of the first subject to be examined; and e) performing attenuation compensation on the photopeak-energy window projection using the generated attenuation map. The method may include additional, less, or alternate functionality, including that discussed elsewhere herein.

In a third aspect, a computer device for single-photon emission computed tomography (SPECT) is provided. The computer device includes at least one processor in communication with at least one memory device. The at least one processor is programmed to: a) store a model trained to generate an attenuation map of a subject being examined; b) receive a scatter-energy window projection of a first subject to be examined; c) execute the model with the scatter-energy window projection of the first subject as an input, wherein the model generates an attenuation map; d) receive a photopeak-energy window projection of the first subject to be examined; and e) perform attenuation compensation on the photopeak-energy window projection using the generated attenuation map. The computer device may include additional, less, or alternate functionality, including that discussed elsewhere herein.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates a system for performing myocardial perfusion SPECT imaging in an imaging environment in accordance with at least one embodiment.

FIG. 2 illustrates an exemplary method for physics- and DL-based method for Tx-less attenuation compensation (PDLAC) for SPECT images in accordance with at least one embodiment.

FIGS. 3A and 3B illustrate some examples of SPECT images and corresponding attenuation maps estimated by the PDLAC method, compared with these from CTAC.

FIG. 4 illustrates a graph of non-inferior statistical testing between PDLAC and CTAC.

FIG. 5 illustrates a graph of the AUC obtained from the CTAC, PDLAC, and NAC methods, in accordance with at least one embodiment.

FIG. 6 illustrates a simplified block diagram of an exemplary computer system for physics- and DL-based method for Tx-less attenuation compensation (PDLAC) for SPECT images.

FIG. 7 illustrates an example configuration of a client system shown in FIG. 6, in accordance with one embodiment of the present disclosure.

FIG. 8 illustrates an example configuration of a server system shown in FIG. 6, in accordance with one embodiment of the present disclosure.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

Advantages—This invention addresses a method to address this issue. This invention is not just expected to address the above laid out issues and make diagnostic SPECT more accurate with low costs and radiation dose, but in addition, could enable diagnostic cardiac SPECT at remote locations, thus further expanding the cardiac SPECT footprint.

The present disclosure generally relates to computer-aided systems and methods of performing single-photon emission computed tomography (SPECT) imaging, and more specifically to performing attenuation compensation in SPECT imaging without the use of a computed tomography (CT) scan. Myocardial perfusion single-photon emission computed tomography (SPECT) (MPS) has an established and well-validated role in improving the diagnosis of coronary artery disease. Attenuation compensation (AC) has been shown to be beneficial for clinical interpretation of single-photon emission computed tomography (SPECT) myocardial perfusion imaging (MPI). However, typical AC methods require the availability of a transmission scan, most often a CT scan in current SPECT scanners, which has multiple disadvantages such as increased radiation dose, high costs, and possibility of misalignment between SPECT and CT scans. Towards addressing this issue, the presented system and methods describe a physics- and deep learning (DL)-based AC (PDLAC) method to perform AC without a separate transmission scan.

However, medical images are acquired for specific clinical tasks. For clinical application of these methods, evaluation on clinical tasks is crucial. Typically, these methods are designed to minimize some fidelity-based criterion between the reconstructed images and some reference image. However, while promising, these methods have failed to improve performance on clinical tasks in SPECT. Thus, for clinical application of these AC methods, the images should be evaluated based on their performance in clinically relevant tasks. To address this issue, the present disclosure outlines a DL-based AC approach that is designed to preserve the observer-related task-specific information, such as detecting perfusion defects on MPI.

The systems described herein are compared with a CT-based AC method (CTAC) and with non-AC (NAC) images. The PDLAC systems and methods described herein are shown to be statistically similar to the CTAC method and was superior to the NAC method for detecting perfusion defects. These results demonstrate the accuracy of the PDLAC method for AC in SPECT described herein.

Attenuation of photons is a major image-degrading artifact that adversely impacts image quality in single-photon emission computed tomography (SPECT). Attenuation compensation (AC) is beneficial for clinical interpretation of SPECT MPI. However, AC methods require an attenuation map, which is typically obtained by an additional CT transmission scan. This has multiple disadvantages such as increased radiation dose, higher costs, and possible misalignment between SPECT and CT scans, which could lead to inaccurate diagnosis. Further, multiple SPECT systems, such as the emerging solid-state-detector-based SPECT systems, often do not have a CT component. Furthermore, deep learning (DL)-based methods have shown significant promise in this direction. However, these methods have typically been evaluated using figures of merit that measure the fidelity between the images reconstructed using the DL-based approach with a reference standard, such as the image reconstructed with the CT-based AC method. However, as described herein, medical images are acquired for specific clinical tasks. Thus, clinical translation of these Tx-less AC methods requires evaluation on clinical tasks. Thus, there is an important need to evaluate these methods specifically on the clinical task for which the images were acquired.

In this context, this disclosure describes a physics- and DL-based method for Tx-less AC in SPECT referred to as physics- and DL-based Tx-less AC method (PDLAC) that accounts for clinical task use of the images.

While this disclosure describes myocardial perfusion, one having skill in the art would understand that analysis of other parts of the body and/or items may be used in concert with the systems and methods described herein.

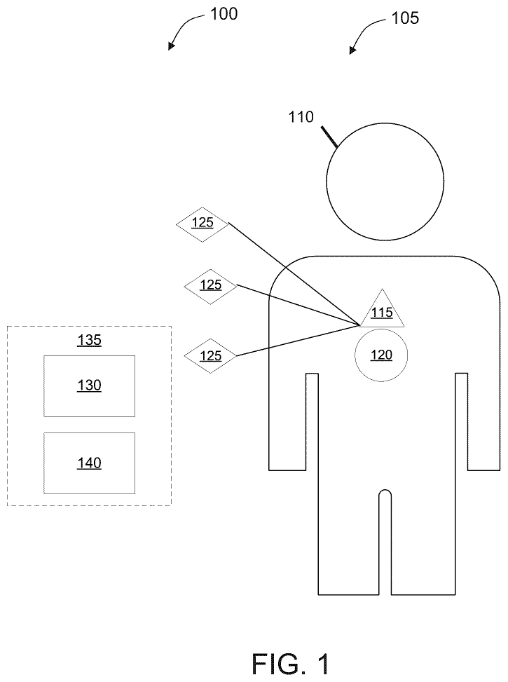

FIG. 1 illustrates a system 100 for performing myocardial perfusion SPECT imaging in an imaging environment 105 in accordance with at least one embodiment.

In the exemplary embodiment, a patient 110 receives a radioactive tracer injection 115 to image their heart 120. The radioactive tracer injection 115 emits gamma(γ)-ray photons 125, which exit the patient's body. A sensor 130, such as, but not limited to, a γ camera, detects the γ-ray photons 125. The sensor 130 is a part of a single-photon emission computed tomography (SPECT) system 135. The SPECT system 135 also includes an attenuation compensation (AC) computer system 140 for performing the analysis described herein to provide images of the patient's heart 120 while the patient's heart 120 is stressed, such as from exercise or particular medications, and at rest.

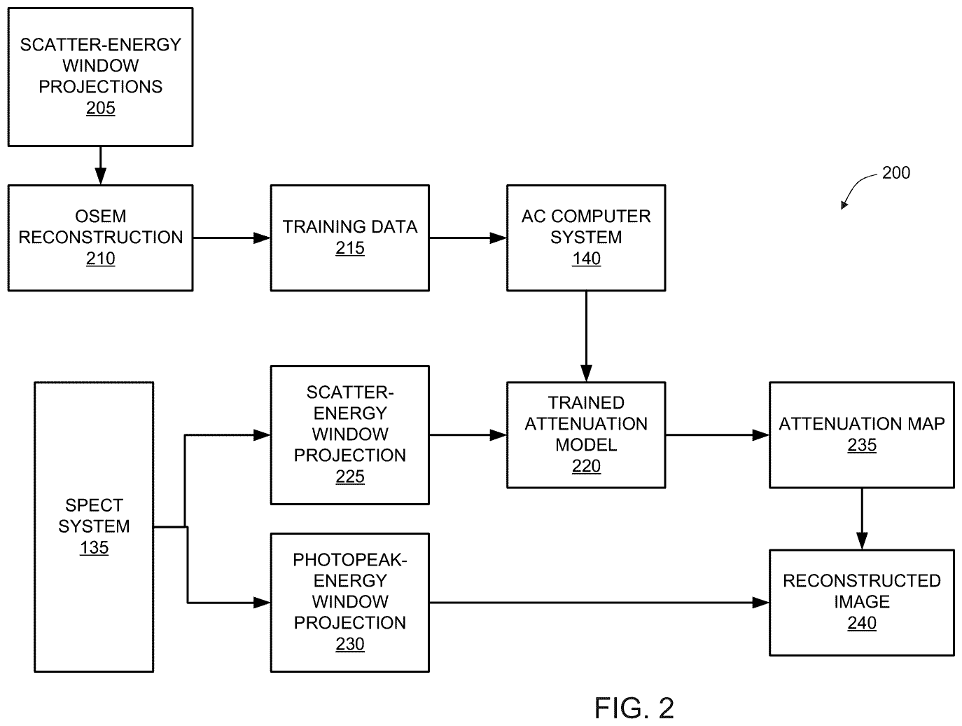

FIG. 2 illustrates an exemplary method 200 for physics- and DL-based method for Tx-less attenuation compensation (PDLAC) for SPECT images in accordance with at least one embodiment. The method 200 is used to provide supervised training to generate attenuation maps from scatter-energy window projections for SPECT images.

In the exemplary embodiments, scatter-energy window projections 205 are reconstructed 210 using an ordered-subsets expectation maximization (OSEM)-based approach. These reconstructed images are used as training data 215. The training data 215 is then input to the AC computer system 140 to generate a trained model 220 for attenuation maps. The trained model 220 is trained to receive scatter-energy window projections 225 from the SPECT system 135. The trained model 220 then generates an attenuation map 235 from the scatter-energy window projection 225. The AC computer system 140 combines the attenuation map with the photopeak-energy window projection 230 to generate the final reconstructed image 240. The final reconstructed image 240 are generated as described below to retain the needed features for visual inspection by a human observer, such as a health care provider.

The overall framework of the PDLAC method 200 uses the fact that scatter-window photons in SPECT contain information to estimate the attenuation coefficients. To use this fact, first, the scatter-energy window projections 205 are reconstructed 210 using an ordered-subsets expectation maximization (OSEM)-based approach. These reconstructions 210 served as initial estimates of an attenuation map. Also, an initial estimate of the activity map was obtained by reconstructing the photopeak (PP)-window projections without any AC using an OSEM-based approach.

In at least one embodiment, a neural network, such as, a Multi-channel Input and Multi-encoder U-Net (McEUN), is trained to segment this initial estimate of attenuation map into several regions, corresponding to different organs in the thoracic region including background, skin and subcutaneous adipose, muscles and organs, lungs, bones, and CT holder. In at least one embodiment, the McEUN consists of three components: an encoder with multi-channel input, an assembly of six decoders, and skip connections with attention gate (AG). In these embodiments, the McEUN is trained to minimize the cross entropy between estimated and ground truth segmentations. In these embodiments, the McEUN is trained with the Adam optimizer and a five-fold cross validation is implemented to prevent overfitting.

The AC computer system 140 performs the training and testing procedures. Predefined attenuation coefficients are then assigned to segmented regions, yielding the final estimated attenuation map. Next, using this attenuation map and the PP energy window projections, the activity map is reconstructed using an OSEM-based AC approach. Finally, following the routine clinical protocol, all reconstructed SPECT images were reorientated into short-axis images and then filtered by a 2-D Butterworth filter with order of 5 and cutoff frequency 0.44 cm⁻¹.

In one embodiment, a sample training data set 215 may be created to include anonymized clinical SPECT/CT stress MPI studies. For this example, patients diagnosed to have normal rest and stress myocardial perfusion function would be categorized as healthy, while patients diagnosed to have an ischemia in the ventricular wall would be referred to as diseased. The MPI scans may be acquired on a SPECT system 135, such as, but not limited to, a GE Discovery NM/CT 670 system following the injection of 99mTc-tetrofosmin. SPECT emission data would be collected in both photopeak (126-154 KeV) 230 and scatter windows (114-126 keV) 225. For training data 215, CT images may also be acquired at 120 kVp on a GE Optima CT 540 system integrated in the GE Discovery NM/CT 670. The AC computer system 140 calculates the CT-defined attenuation maps from the CT scans such as by using a bi-linear model.

The attenuation maps used for training were segmented into background, skin and subcutaneous adipose, muscles and organs, lungs, bones, and CT holder, such as by using a Markov random field-based method. The dataset 215 could be divided into the training and testing datasets. Using the training data 215, the network (aka model 220) was trained as described in sub-section above.

For training the model 220 and to conduct the task-based evaluation study with the testing dataset 215, the knowledge of the absence/presence and location of the defects is needed. Therefore, the training dataset 215 may instead use a strategy of introducing synthetic cardiac defects in healthy images. In at least one embodiment, 27 types of clinically realistic defects with three radial extents (30, 60, and 90 degrees around the left ventricular (LV) wall), three severities (10%, 25%, and 50% less activity than remainder of the myocardium), and at three locations (anterior, inferior, and lateral walls of the LV) are used to test the training dataset 215. In at least one embodiment, these synthetic defects are added in 71 of the 140 healthy samples in the test dataset 215. To insert the defect, the AC computer system 140 first segments the LV from the cardiac short-axis images, such as by using segment software. Then the AC computer system 140 applies the defect masks of 27 defect types with desired angular extent at desired location that was created for each patient sample. Using these masks, the training dataset 215 may be generated with 27×71=1,917 defect-present samples in both PP and scatter energy windows.

In at least one embodiment, the PDLAC method 200 is compared with CT-based AC method (CTAC) and with non-AC (NAC)-based approaches. To obtain the CTAC images for the test dataset, the SPECT projections with and without defects were reconstructed with AC using CT scans using an OSEM-based approach. The NAC images could be obtained by reconstructing the SPECT images using an OSEM-based approach but without any AC.

The comparison with the CTAC and NAC approaches allows for the evaluation of the performance of the PDLAC method 200 on the task of detecting myocardial perfusion defects using a model observer. While ideally such evaluation should be performed with human observers, this can be time consuming and tedious. Model observers provide an easy-to-use in silico approach to perform such evaluation and identify methods for evaluation with human observers. Thus, multiple studies use model observers to evaluate imaging systems and methods.

Using the test statistics, the AC computer system 140 performs a ROC analysis and the area under the ROC curve (AUC) is calculated using LABROC4 program. The AUC obtained from PDLAC method is compared to that obtained from the CTAC and NAC methods. This may include testing for similarity between PDLAC and CTAC methods by using noninferiority statistical testing. The noninferiority margin Δ may be set to be 3% of AUC obtained from CTAC method.

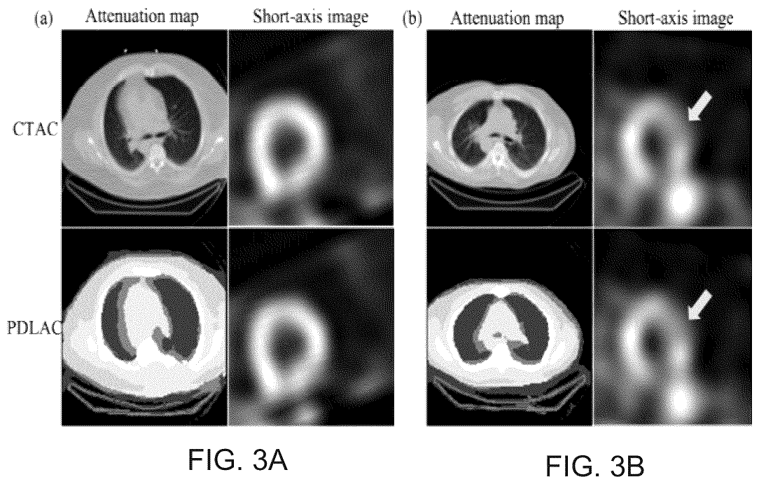

FIGS. 3A and 3B illustrate some examples of SPECT images and corresponding attenuation maps estimated by the PDLAC method 200, compared with these from CTAC. FIG. 3A illustrates a defect absent example. FIG. 3B illustrates a defect present example.

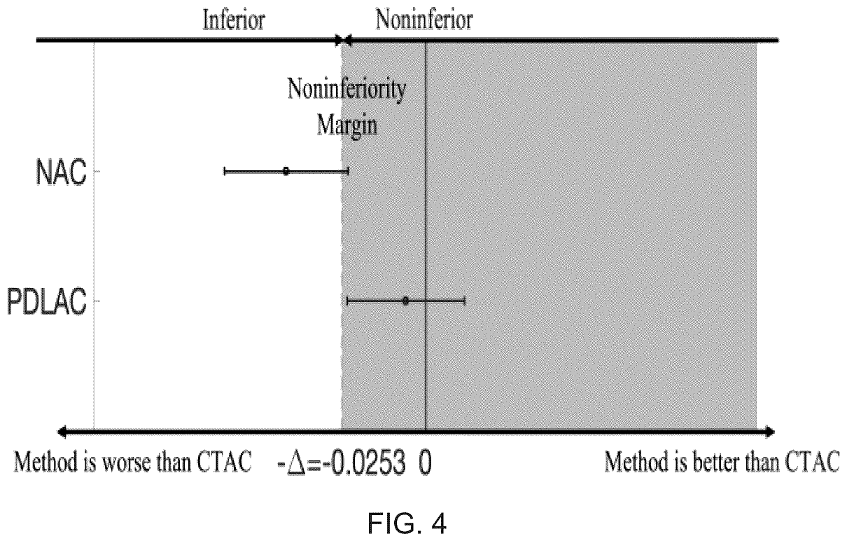

FIG. 4 illustrates a graph of non-inferior statistical testing between PDLAC and CTAC. In at least one embodiment, NAC is treated as a placebo.

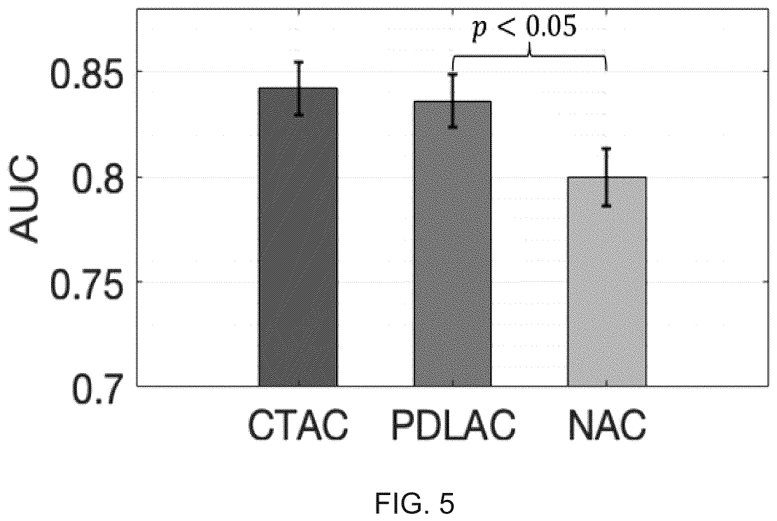

FIG. 5 illustrates a graph of the AUC obtained from the CTAC, PDLAC, and NAC methods, in accordance with at least one embodiment. As shown in the graph, the physics- and learning-based transmission-less AC method for SPECT yielded statistically noninferior performance compared to the standard AC method, as evaluated on the clinically relevant task of detecting myocardial perfusion defects using retrospective clinical data. Accordingly, the task-based evaluation of a physics- and learning-based transmission-less AC method for SPECT using a retrospective clinical study with single-center data and with a model observer showed that the method was statistically noninferior to a standard CT-based AC method on the task of detecting myocardial perfusion defects with the AUC difference within a pre-defined margin of 3% of AUC obtained from CT-based AC method.

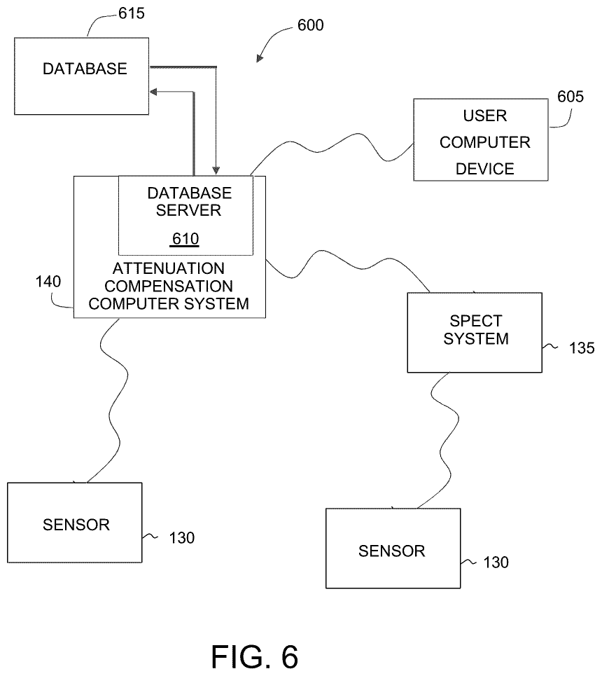

FIG. 6 illustrates a simplified block diagram of an exemplary computer system 600 for physics- and DL-based method for Tx-less attenuation compensation (PDLAC) for SPECT images. In the exemplary embodiment, computer system 600 may be used for training and using a model for performing attenuation compensation without CT imaging. As described below in more detail, an AC computer device 140 may be configured to a) store a model 220 trained to generate an attenuation map 235 of a subject 110 being examined; b) receive a scatter-energy window projection 225 of a first subject 110 to be examined; c) execute the model 220 with the scatter-energy window projection 225 of the first subject 110 as an input, wherein the model 220 generates an attenuation map 235; d) receive a photopeak-energy window projection 230 of the first subject 110 to be examined; and e) perform attenuation compensation on the photopeak-energy window projection 230 using the generated attenuation map 235.

In the exemplary embodiment, user computer devices 605 are computers that include a web browser or a software application, which enables user computer devices 605 to access remote computer devices, such as the AC computer device 140, using the Internet or other network. More specifically, user computer devices 605 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. User computer devices 605 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

A database server 610 may be communicatively coupled to a database 315 that stores data. In one embodiment, database 615 may include the training data 215, the trained model 220 for AC, attenuation maps 235, and reconstructed images 240 (all shown in FIG. 2). In the exemplary embodiment, database 615 may be stored remotely from the AC computer device 140. In some embodiments, database 615 may be decentralized. In the exemplary embodiment, the user may access database 615 via user computer device 605 by logging onto AC computer device 140, as described herein.

Attenuation compensation (AC) computer device 140 may be communicatively coupled with one or more sensors 130, SPECT system 135, and user computer device 605. In some embodiments, AC computer device 140 may be associated with, or is part of a computer network associated with a SPECT system 135 or individual scanner. In other embodiments, AC computer device 140 may be associated with a third party and is merely in communication with the SPECT system 135. More specifically, the AC computer device 140 is communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. The AC computer device 140 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices. In the exemplary embodiment, the AC computer device 140 hosts an application or website that allows the user to perform analysis of SPECT images including attenuation compensation without CT scans.

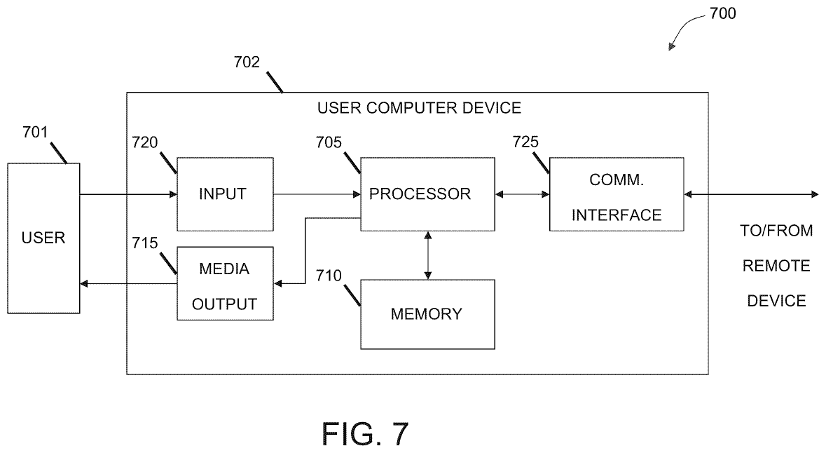

FIG. 7 depicts an exemplary configuration of the computer devices shown in FIGS. 1, 2, and 6, in accordance with one embodiment of the present disclosure. User computer device 702 may be operated by a user 701. User computer device 702 may include, but is not limited to, AC computer device 140 (shown in FIG. 1) and user computer device 605 (shown in FIG. 6). User computer device 702 may include a processor 705 for executing instructions. In some embodiments, executable instructions are stored in a memory area 710. Processor 705 may include one or more processing units (e.g., in a multi-core configuration). Memory area 710 may be any device allowing information such as executable instructions and/or transaction data to be stored and retrieved. Memory area 710 may include one or more computer readable media.

User computer device 702 may also include at least one media output component 715 for presenting information to user 701. Media output component 715 may be any component capable of conveying information to user 701. In some embodiments, media output component 715 may include an output adapter (not shown) such as a video adapter and/or an audio adapter. An output adapter may be operatively coupled to processor 705 and operatively coupleable to an output device such as a display device (e.g., a cathode ray tube (CRT), liquid crystal display (LCD), light emitting diode (LED) display, or “electronic ink” display) or an audio output device (e.g., a speaker or headphones).

In some embodiments, media output component 715 may be configured to present a graphical user interface (e.g., a web browser and/or a client application) to user 701. A graphical user interface may include, for example, a reconstructed image 240 (shown in FIG. 2). In some embodiments, user computer device 702 may include an input device 720 for receiving input from user 701. User 701 may use input device 720 to, without limitation, select and/or enter one or more scatter-energy window projections 225 and/or photopeak-energy window projections 230.

Input device 720 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 715 and input device 720.

User computer device 702 may also include a communication interface 725, communicatively coupled to a remote device such as AC computer device 140, SPECT system 135, or sensor 130 (all shown in FIG. 1). Communication interface 725 may include, for example, a wired or wireless network adapter and/or a wireless data transceiver for use with a mobile telecommunications network.

Stored in memory area 710 are, for example, computer readable instructions for providing a user interface to user 701 via media output component 715 and, optionally, receiving and processing input from input device 720. A user interface may include, among other possibilities, a web browser and/or a client application. Web browsers enable users, such as user 701, to display and interact with media and other information typically embedded on a web page or a website from AC computer system 140. A client application allows user 701 to interact with, for example, sensors 130. For example, instructions may be stored by a cloud service, and the output of the execution of the instructions sent to the media output component 715.

Processor 705 executes computer-executable instructions for implementing aspects of the disclosure. In some embodiments, the processor 705 is transformed into a special purpose microprocessor by executing computer-executable instructions or by otherwise being programmed. For example, the processor 705 may be programmed with the instructions such as those described in FIG. 2.

In some embodiments, user computer device 702 may include, or be in communication with, one or more sensors, such as sensor 130. User computer device 702 may be configured to receive data from the one or more sensors and store the received data in memory area 710. Furthermore, user computer device 702 may be configured to transmit the sensor data to a remote computer device, such as AC computer device 140, through communication interface 725.

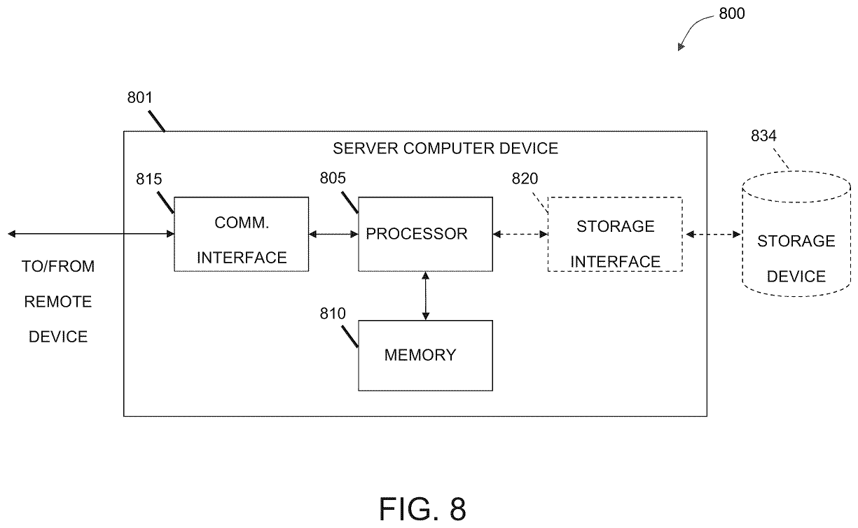

FIG. 8 illustrates an example configuration of the server system shown in FIG. 6, in accordance with one embodiment of the present disclosure. Server computer device 801 may include, but is not limited to, AC computer device 140 (shown in FIG. 1) and database server 615 (shown in FIG. 6). Server computer device 801 also includes a processor 805 for executing instructions. Instructions may be stored in a memory area 810. Processor 805 may include one or more processing units (e.g., in a multi-core configuration).

Processor 805 is operatively coupled to a communication interface 815 such that server computer device 801 is capable of communicating with a remote device such as another server computer device 801, AC computer device 140, sensors 130, or user computer device 605 (shown in FIG. 6). For example, communication interface 815 may receive requests from user computer devices 605 and information from sensors 130 (shown in FIG. 1) via the Internet, as illustrated in FIG. 6.

Processor 805 may also be operatively coupled to a storage device 834. Storage device 834 is any computer-operated hardware suitable for storing and/or retrieving data, such as, but not limited to, data associated with database 615 (shown in FIG. 6). In some embodiments, storage device 834 is integrated in server computer device 801. For example, server computer device 801 may include one or more hard disk drives as storage device 834. In other embodiments, storage device 834 is external to server computer device 801 and may be accessed by a plurality of server computer devices 801. For example, storage device 834 may include a storage area network (SAN), a network attached storage (NAS) system, and/or multiple storage units such as hard disks and/or solid-state disks in a redundant array of inexpensive disks (RAID) configuration.

In some embodiments, processor 805 is operatively coupled to storage device 834 via a storage interface 820. Storage interface 820 is any component capable of providing processor 805 with access to storage device 834. Storage interface 820 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 805 with access to storage device 834.

Processor 805 executes computer-executable instructions for implementing aspects of the disclosure. In some embodiments, the processor 805 is transformed into a special purpose microprocessor by executing computer-executable instructions or by otherwise being programmed. For example, the processor 805 is programmed with instructions such as those disclosed in FIG. 2.

The computer-implemented methods and processes described herein may include additional, fewer, or alternate actions, including those discussed elsewhere herein. The present systems and methods may be implemented using one or more local or remote processors, transceivers, and/or sensors (such as processors, transceivers, and/or sensors mounted on computer systems or mobile devices, or associated with or remote servers), and/or through implementation of computer-executable instructions stored on non-transitory computer-readable media or medium. Unless described herein to the contrary, the various steps of the several processes may be performed in a different order, or simultaneously in some instances.

Additionally, the computer systems discussed herein may include additional, fewer, or alternative elements and respective functionalities, including those discussed elsewhere herein, which themselves may include or be implemented according to computer-executable instructions stored on non-transitory computer-readable media or medium.

In the exemplary embodiment, a processing element may be instructed to execute one or more of the processes and subprocesses described above by providing the processing element with computer-executable instructions to perform such steps/sub-steps, and store collected data (e.g., vehicle profiles, etc.) in a memory or storage associated therewith. This stored information may be used by the respective processing elements to make the determinations necessary to perform other relevant processing steps, as described above.

The aspects described herein may be implemented as part of one or more computer components, such as a client device, system, and/or components thereof, for example. Furthermore, one or more of the aspects described herein may be implemented as part of a computer network architecture and/or a cognitive computing architecture that facilitates communications between various other devices and/or components. Thus, the aspects described herein address and solve issues of a technical nature that are necessarily rooted in computer technology.

A processor or a processing element may be trained using supervised or unsupervised machine learning, and the machine learning program may employ a neural network, which may be a convolutional neural network, a deep learning neural network, a reinforced or reinforcement learning module or program, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, object statistics and information, traffic timing, previous trips, and/or actual timing. The machine learning programs may utilize deep learning algorithms that may be primarily focused on pattern recognition and may be trained after processing multiple examples. The machine learning programs may include Bayesian Program Learning (BPL), voice recognition and synthesis, image or object recognition, optical character recognition, and/or natural language processing-either individually or in combination. The machine learning programs may also include natural language processing, semantic analysis, automatic reasoning, and/or machine learning.

Supervised and unsupervised machine learning techniques may be used. In supervised machine learning, a processing element may be provided with example inputs and their associated outputs and may seek to discover a general rule that maps inputs to outputs, so that when subsequent novel inputs are provided the processing element may based upon the discovered rule, accurately predict the correct output. In unsupervised machine learning, the processing element may be required to find its own structure in unlabeled example inputs. In one embodiment, machine learning techniques may be used to determine how different photons exit the body based upon body structures.

Based upon these analyses, the processing element may learn how to identify characteristics and patterns that may then be applied to analyzing image data, model data, and/or other data. For example, the processing element may learn, to identify trends of locations based on photon vectors and locations. The processing element may also learn how to identify trends that may not be readily apparent based upon collected data, such as trends that identifying optimal placement of treatments in the body relative to tumors.

The singular forms “a,” “an,” and “the” include plural references unless the context clearly dictates otherwise.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

As used herein, the term “database” may refer to either a body of data, a relational database management system (RDBMS), or to both, and may include a collection of data including hierarchical databases, relational databases, flat file databases, object-relational databases, object oriented databases, and/or another structured collection of records or data that is stored in a computer system. The above examples are not intended to limit in any way the definition and/or meaning of the term database. Examples of RDBMS's include, but are not limited to, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

A computer program of one embodiment is embodied on a computer-readable medium. In an example, the system is executed on a single computer system, without requiring a connection to a server computer. In a further example embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

As used herein, the terms “processor” and “computer” and related terms, e.g., “processing device”, “computing device”, and “controller” are not limited to just those integrated circuits referred to in the art as a computer, but broadly refers to a microcontroller, a microcomputer, a programmable logic controller (PLC), an application specific integrated circuit (ASIC), and other programmable circuits, and these terms are used interchangeably herein. In the embodiments described herein, memory may include, but is not limited to, a computer-readable medium, such as a random-access memory (RAM), and a computer-readable non-volatile medium, such as flash memory. Alternatively, a floppy disk, a compact disc-read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

Further, as used herein, the terms “software” and “firmware” are interchangeable and include any computer program storage in memory for execution by personal computers, workstations, clients, servers, and respective processing elements thereof.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device, and a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Furthermore, as used herein, the term “real-time” refers to at least one of the time of occurrence of the associated events, the time of measurement and collection of predetermined data, the time for a computing device (e.g., a processor) to process the data, and the time of a system response to the events and the environment. In the embodiments described herein, these activities and events may be considered to occur substantially instantaneously.

Exemplary embodiments of systems and methods for securely navigating traffic lights are described above in detail. The systems and methods of this disclosure though, are not limited to only the specific embodiments described herein, but rather, the components and/or steps of their implementation may be utilized independently and separately from other components and/or steps described herein.

Although specific features of various embodiments may be shown in some drawings and not in others, this is for convenience only. In accordance with the principles of the systems and methods described herein, any feature of a drawing may be referenced or claimed in combination with any feature of any other drawing.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a programmable logic unit (PLU), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above examples are exemplary only, and thus are not intended to limit in any way the definition and/or meaning of the term processor and processing device.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for single-photon emission computed tomography (SPECT) comprising a computer device comprises at least one processor in communication with at least one memory device, wherein the at least one processor is programmed to:
store a model trained to generate an attenuation map of a subject being examined;
receive a scatter-energy window projection of a first subject to be examined;
execute the model with the scatter-energy window projection of the first subject as an input, wherein the model generates an attenuation map;
receive a photopeak-energy window projection of the first subject to be examined; and
perform attenuation compensation on the photopeak-energy window projection using the generated attenuation map.

2. The system of claim 1, wherein the at least one processor is further programmed to train the model with a plurality of scatter-energy window projections.

3. The system of claim 2, wherein the at least one processor is further programmed to perform ordered-subsets expectation maximization (OSEM) reconstruction on the plurality of scatter-energy window projections.

4. The system of claim 1, wherein the scatter-energy window projection and the photopeak-energy window projection are received from the same SPECT system.

5. The system of claim 1, wherein the scatter-energy window projection and the photopeak-energy window projection are received simultaneously.

6. The system of claim 1, wherein the attenuation compensation on the photopeak-energy window projection is performed with an OSEM approach.

7. The system of claim 1, wherein the photopeak-energy window projection is received from a SPECT system without a computer tomography (CT) scanner.

8. The system of claim 1, wherein the at least one processor is further programmed to execute the model for a plurality of photopeak-energy window projections.

9. The system of claim 8, wherein the plurality of photopeak-energy window projections is a part of a video.

10. The system of claim 1, wherein the photopeak-energy window projection was taken during a myocardial perfusion.

11. A method for single-photon emission computed tomography (SPECT), the method implemented by a computer device comprising at least one processor in communication with one or more memory devices, the method comprises:
storing a model trained to generate an attenuation map of a subject being examined;
receiving a scatter-energy window projection of a first subject to be examined;
executing the model with the scatter-energy window projection of the first subject as an input, wherein the model generates an attenuation map;
receiving a photopeak-energy window projection of the first subject to be examined; and
performing attenuation compensation on the photopeak-energy window projection using the generated attenuation map.

12. The method of claim 11 further comprising training the model with a plurality of scatter-energy window projections.

13. The method of claim 12 further comprising performing ordered-subsets expectation maximization (OSEM) reconstruction on the plurality of scatter-energy window projections.

14. The method of claim 11, wherein the scatter-energy window projection and the photopeak-energy window projection are received from the same SPECT system.

15. The method of claim 11, wherein the scatter-energy window projection and the photopeak-energy window projection are received simultaneously.

16. The method of claim 11, wherein the attenuation compensation on the photopeak-energy window projection is performed with an OSEM approach.

17. The method of claim 11, wherein the photopeak-energy window projection is received from a SPECT system without a computer tomography (CT) scanner.

18. The method of claim 11 further comprising executing the model for a plurality of photopeak-energy window projections.

19. The method of claim 18, wherein the plurality of photopeak-energy window projections is a part of a video.

20. The method of claim 11, wherein the photopeak-energy window projection was taken during a myocardial perfusion.
